(19) United States
(12) Patent Application Publication
(10) Pub. No.: US 2011/0053117 A1
(43) Pub. Date: Mar. 3, 2011

(54) Root Canal Filling Material

(76) Inventors: Jürgen Engelbrecht, Hamburg (DE); Gunther Groeger, Hamburg (DE)

(21) Appl. No.: 12/664,803

(22) PCT Filed: Jun. 26, 2008
(86) PCT No.: PCT/EP2008/005227

§ 371 Date: Mar. 16, 2010

(30) Foreign Application Priority Data
Jun. 26, 2007 (DE) 102007029640.3

(51) Int. Cl. A61C 5/02; A61K 6/083; A61C 5/11

(52) U.S. Cl. 433/224; 523/117; 433/228.1

(57) ABSTRACT

The invention describes polymerisable compositions which contain higher molecular weight di(meth)acrylates, curing agents and X-ray-opacifying additives, which result in fully cured, readily removable materials and which are especially suitable for filling and sealing root canals.

The invention describes polymerisable compositions which contain higher molecular weight di(meth)acrylates, curing agents and X-ray-opacifying additives, which result in fully cured readily removable materials and which are especially suitable for filling and sealing root canals.

### PRIOR ART

Root canal filling materials are used for filling the prepared root canal. Preparation procedures for such fillings are normally carried out “blind”. That means that the result of preparatory measures and the filling itself cannot be directly assessed visually. A disadvantage of most filling materials for root canal fillings is that they shrink on hardening in the root canal. This can in turn result in the occurrence of shrinkage-induced gaps. As a result of such gaps it is possible, for example, for secretion from the root apex to penetrate the tooth cavity, with the result that a breeding ground for bacteria is created.

During a root canal treatment, first of all the pulp tissue is removed. Then the root canal lumen is prepared using standard instruments. It is often the case that the root canal is not immediately given a final filling, but is filled with an antimicrobial calcium hydroxide paste for a period of from a few days to several weeks. A standard gutta-percha point (of a shape congruent with the last instrument size used for the preparation) is fitted into the prepared root canal. The seating of that point (master-point) is often monitored by means of X-ray monitoring. The point and/or the canal wall are then coated with a root canal filling material and the point is introduced into the root canal again. After the introduction of a first point, any gaps that remain can be reduced by subsequent insertion of needle-like condensation instruments. By means of such condensing instruments, the deformed point and the filling material are adapted to the canal wall. It is possible then to insert further points in order that the canal wall, which is often still irregular, is tightly filled. That procedure is very laborious, however.

The known filling materials for teeth or root canals require, as described above, very complicated and laborious processing methods in order to avoid the risk of the formation of shrinkage-induced gaps between the filling and the tooth or the root canal. Such a complicated and time-consuming procedure is a considerable burden on the patient and gives rise to considerable expense on account of the time-consuming and labour-intensive treatment method.

A sealing material for root canal filling which is widely used at present is a material containing zinc oxide and eugenol as main components. Although the sealing composition based on zinc oxide/eugenol for root canal filling is able to fill up the space between the wall of the root canal and the gutta-percha point, it does not have the adhesive properties in respect of both the wall of the root canal and the gutta-percha point. That accordingly entails the shortcoming that its capacity to seal the root canal is not sufficient from a clinical standpoint. In addition, eugenol is harmful to living things and therefore involves a safety problem.

Commercially available products exist that employ as sealing composition for root canal fillings a dental glass ionomer cement which is used for filling or for cementing in dentistry. Such a glass ionomer cement, used for root canal fillings, is able to adhere to the tooth and can also adhere to the gutta-percha point. Consequently, the dental glass ionomer cement is excellently suitable for sealing the root canal and, by virtue of its high degree of biocompatibility, is to be regarded as toxicologically harmless. The dental glass ionomer cement is nevertheless not generally used for the reasons described below.

When the glass ionomer cement is used as sealing agent, not only does it have high adhesion properties in respect of the gutta-percha point and dentine, but it also has high strength (the compressive strength is about 110 MPa). As a result, in the event of complications it is difficult to remove the gutta-percha point and the glass ionomer cement by excision with a reamer or a file, because the root canal filling cannot be removed again using a drill without the risk of penetrating the canal wall and its adverse consequences. It is then therefore frequently the case that tooth extraction is unavoidable.

In general, the root canal has a complicated shape and there may be three or four root canals present, depending upon the type of tooth. In addition, the shape includes different variations depending upon the teeth in question, such as a flat shape, a cylindrical shape or a curved shape. For those reasons, even when the treatment has been carried out using a gutta-percha point and the sealing agent for the root canal filling as described above, it is difficult fully to seal the root canal in every detail in order to prevent the penetration of bacteria, leading to the possibility of toothache or swelling of the gums caused by the penetration of bacteria or the like. In such a case, it is necessary to perform a root canal treatment again. In other words, it is necessary to remove the gutta-percha point and the sealing material—each of which has been introduced into the root canal—using a reamer or a file and to re-introduce the gutta-percha point using the lateral condensation method described above or some other method.

Root filling materials are often introduced into the root canal using a needle. On account of the dimensions of the needle (and of the root canal) it is necessary for the material used to have a sufficiently low viscosity. Alternatively, root canal filling materials can be introduced using a lentulo or gutta-percha tip. Accordingly, the viscosity must be sufficiently low for a film to be formed. In addition, it must be possible for the material to be able to penetrate into the dentine canals of the root canal.

Because the application of root canal filling materials has to be monitored using X-ray images during the introduction phase in order to prevent introduction beyond the apex of the root canal, a content of radio-opaque fillers in the composition of the root canal filling material is indicated. WO 2002/13767 describes root canal filling materials which, in the form of a paste/paste system, are composed of addition-polymerising equivalent amounts of low molecular weight diamines and low molecular weight diacrylates. It is optional in this case to use a reactive diluent for regulating the viscosity.

Nothing else is mentioned apart from its being sufficiently X-ray-visible and setting only after 30 minutes.

Root canal filling materials based on primary monoamines and/or secondary diamines and diepoxides (U.S. Pat. No. 5,624,976), diacrylates (WO 2002/13767) or bisacrylamides (WO 2002/13768) are distinguished by advantages such as a long processing time, high radio-opacity, low shrinkage, low solubility and good sealing properties [Int. Endod. J. 2003, January 36(1): 54-63; Int. Endod. J. 1999 September 32(5): 415-418; Aust. Endod. J. 2001 April 27(1): 33-5; J. Endod. 1999 March 25(3): 172-7)].

The use of low molecular weight amines in root canal filling materials nevertheless also results in serious disadvantages. Cytotoxic effects resulting from the amines' being dissolved out from the root canal are frequently observed. The polymerising compositions from WO 2002/13767 exhibit considerable solubility, thus exacerbating the problem of cytotoxicity. In addition, the high vapour pressure and the resulting high penetration rate of low molecular weight amines through plastics packagings detracts from use in industrial applications [Endod. Dent. Traumatol. 1998 August 14(4): 182-5; Endod. Traumatol. 2000 December 16(6): 287-90; J. Endod. 2000 August 26(8): 462-5; J. Endod. 2000 April 26(4): 228-9).

Polyamino esters, such as described in WO 2002/13767, are highly viscous and require the use of a not inconsiderable proportion of a reactive diluent in order to lower the viscosity. Such reactive diluents cannot be polymerised by addition polymerisation and require the presence of a polymerisation initiator.

US 2002/0143108 discloses macro-monomers which are obtained in a two-step reaction by reaction of diepoxides with secondary diamines. The intermediate prepolymer reacts in a second step with a 2,3-epoxypropyl(meth)acrylate and was not isolated. Neither was any further information published which indicated that the prepolymer would be suitable for providing an amino-terminated prepolymer having a viscosity of <100 Pas at 23° C.

U.S. Pat. No. 4,449,938 and EP 0 864 312 describe organopolysiloxane-based two-component materials which fully cure at room temperature and are used as root canal filling material. Those materials are advantageous on account of their very low shrinkage during full cure and the ease with which they can be removed again. Their relatively high hydrophobicity and lack of binding ability to the root canal wall are disadvantageous, however.

Various root canal filling materials which contain, for example, swellable hydrophilic acrylates and methacrylates have been described. For example, U.S. Pat. No. 3,925,895 describes the use of monomers (such as, for example, 2-hydroxyethyl methacrylate (HEMA)) which are mixed with initiators immediately before application to the root canal, which monomers then fully cure in situ. The disadvantage of such mixtures lies, on the one hand, in that they are based predominantly on low molecular weight monomethacrylates which generally exhibit a low conversion rate, which is toxicologically harmful. A further disadvantage is their high degree of polymerisation shrinkage. On the other hand, the swelling to be compensated is considerably too high at 5 to 25% and can result in excessive hydraulic pressures.

The problem of the present invention was to find root canal filling materials
having sufficiently low viscosity, a sufficiently long processing time and sufficient X-ray visibility for monitored introduction into the root canal,
having sufficiently good mechanical properties in order to withstand the necessary loads, but being sufficiently capable of being cut or milled in order to be removable again, if necessary, without problems, the materials being easier to cut and penetrate than the wall of the root canal in order that the wall itself is not penetrated,
having a volumetric behaviour such that the quality of sealing is high during and after setting in order that marginal gaps and accordingly the possibility of bacterial penetration are avoided, and
having low solubility and good compatibility.

The problem has been solved according to the invention by compositions in accordance with the claims.

The composition solving the problem of the invention is very suitable as a root canal filling material and according to the invention is especially characterised in that it comprises one or more higher molecular weight di(meth)acrylates, one or more curing agents and one or more X-ray-opacifying additives. The composition is sufficiently fluid prior to polymerisation and after polymerisation yields readily machinable shaped bodies providing good sealing. The root canal filling material according to the invention has the great advantage that by virtue of the relatively low polymerisation shrinkage and the balanced hydrophilicity of the higher molecular weight monomers it is possible to achieve better marginal sealing of the root canal filling. In comparison with other methacrylates-based root canal filling materials that contain short-chain hydrophilic or long-chain degradable monomers, the volumetric behaviour of the root canal filling material according to the invention is significantly improved.

The acrylates suitable for use as monomer according to the invention have, for example, the general formula (I):

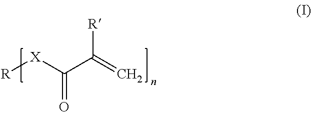

wherein X is an oxygen atom or a single bond, R is a (meth)acryl-free organic radical, a dimer, an oligomer or a polymer which is composed of up to 1000 monomeric units, and R′ is hydrogen, halogen, an alkyl radical, preferably C₁-C₆alkyl, a substituted alkyl radical, preferably substituted C₁-C₆alkyl, or a cyano radical, and n is a whole number from 1 to 6.

When n=1, a relatively low polymerisation rate can be achieved and accordingly a less strong material obtained, if desired. When n=3 to 6, a high degree of cross-linking can be achieved and accordingly a stronger material obtained, if that is desirable. For most applications a diacrylate with n=2 is most suitable as root canal filling material, because it is therewith possible to achieve an adequate polymerisation rate without the material's becoming too strong.

Preferably, R is an organic radical such as, for example, alkyl, aryl, cycloalkyl or polyether, polyurethane, polyester, glycol, polyglycol, which can be substituted by carboxylic acid, phosphoric acid or other acids, and salts thereof.

In a preferred embodiment, n=2, that is to say a diacrylate having the general formula (II):

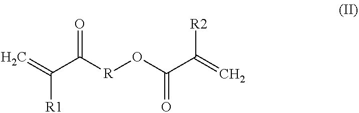

R1 and R2 each independently of the other can be a hydrogen atom, a halogen atom, an optionally substituted alkyl group or a cyano group. Preferably R1 and R2 are identical.

More preferably, R1 and/or R2 are a hydrogen atom or a C₁-C₆alkyl group, which can optionally be substituted by a hydroxy group, a C₁-C₆alkoxy group, an amino group or by a halogen atom. More especially, R1 and/or R2 are a hydrogen atom or a methyl group; most preferably, R1 and R2 are a hydrogen atom or a methyl group, that is to say a di(meth)acrylate.

R is preferably an organic radical having an average molecular weight of at least 400. More preferably, R is alkyl, alkoxy, aryl, alkylaryl, alkoxyaryl, polyalkoxy and polyalkoxyaryl, each of which can be substituted by one or more groups selected from alkyl, alkoxy, polyalkoxy, carboxy and phosphate. More especially, R has one or more polyalkoxy groups of the formula —(O—(CH₂)ₓ)_{y}—, wherein x is a whole number from 1 to 6, preferably 2, that is to say R contains one or more polyethylene groups, and y is a whole number from 1 to 1000, preferably from 1 to 200, especially from 2 to 50, more especially from 5 to 40, most especially from 10 to 30.

The root canal filling material of the invention can contain, for example, additionally dimeric and/or oligomeric and/or prepolymeric polyester and/or polyether (meth)acrylates, urethane (meth)acrylates and polyglycol (meth)acrylates and also unsaturated polyesters.

Preferred polymerisable di(meth)acrylates according to the invention include, for example, polyethylene glycol dimethacrylate having from 10 to 30 ethylene oxide repeating units and/or ethoxylated bisphenol dimethacrylate having from 10 to 30 ethylene oxide repeating units.

Examples are polyethylene glycol 600 dimethacrylate (MW 754), polyethylene glycol 600 diacrylate (MW 770) from Cray Valley, polyalkylene glycol dimethacrylate EP100 DMA (MW 1114) from Bisomer, ethoxylated bisphenol A dimethacrylate E(10)BADMA (MW 804) from Bisomer, ethoxylated bisphenol A dimethacrylate E(30)BADMA (MW 1678), longer chain aliphatic urethane diacrylates, longer chain aliphatic urethane dimethacrylates, aliphatic polyester urethane diacrylates, aliphatic polyester urethane dimethacrylates, polybutadiene dimethacrylate, poly-methylpolysiloxane dimethacrylate or mixtures thereof. Preferably the mixtures according to the invention are formulated so as to be sufficiently fluid for the application.

According to the invention, higher molecular weight di(meth)acrylates comprise at least 10, preferably 20 and especially 30 hydrophilic units.

The higher molecular weight di(meth)acrylates used should preferably have an average molecular weight of more than 600, preferably of at least 750. Preferably, they are present in the polymerisable composition in amounts of from 2 to 90% by weight, especially in amounts of from 10 to 50% by weight, more especially in amounts of from 15 to 30% by weight. Preference is given to di(meth)acrylates having hydrophilic units such as, for example, —CH₂—O— or —CH₂—CH₂—O— and/or OH—, NH₂—, groups. Special preference is given to —CH₂—CH₂—O— units.

It is preferable to use di(meth)acrylates having a plurality of hydrophilic units. Preference is given to compounds having 10 hydrophilic units, especially having 20 hydrophilic units, more especially having 30 hydrophilic units.

It is further preferable for the polymerisable composition according to the invention to contain at least two different higher molecular weight di(meth)acrylates.

According to the invention, the (average) molecular weight of the present compounds is given as weight-averaged molecular weight.

The polymerisable (meth)acrylates according to the invention can contain low-viscosity and/or high-viscosity higher molecular weight di(meth)acrylates.

For adjusting the viscosity and/or the hydrophilicity, in addition to the higher molecular weight di(meth)acrylate(s) in the composition it is also possible for one or more lower molecular weight di(meth)acrylates to be present in the compositions according to the invention. Preferably, the lower molecular weight di(meth)acrylates have a molecular weight of less than 600, especially less than 400 and more especially less than 300.

The agent(s) according to the invention for adjusting the viscosity and/or the hydrophilicity preferably have a viscosity of up to 100 mPa·s, especially from 6 to 50 mPa·s.

By the choice of the viscosity of the di(meth)acrylate(s), it is possible for the composition according to the invention both to be diluted and to achieve its necessary flexibility.

Preferred lower molecular weight di(meth)acrylates are, for example, glycerol dimethacrylate, sorbitol dimethacrylate, ethylene glycol dimethacrylate, triethylene glycol dimethacrylate, tetraethylene glycol dimethacrylate, butanediol dimethacrylate, hexanediol dimethacrylate, decanediol dimethacrylate, dodecanediol dimethacrylate, dimethacryl-bispropyl-tetramethyldisiloxane or mixtures thereof. They are preferably present in the composition with a content of less than 30% by weight, special preference being given to a content of less than 18% by weight.

The root filling material can be cold-polymerised and/or photopolymerised.

As curing agents (catalysts) for cold-polymerisation there can be used systems that yield one or more free radicals, such as, for example, dibenzoyl peroxide or bisdichlorobenzoyl peroxide together with activators such as, for example, with amines such as N,N-dimethyl-sym.-xylidine or N,N-dimethyl-p-toluidine or with copper naphthenate or the like. It is also possible to use malonyl sulfamides, as described in EP 0 059 451, or CH-active compounds having an auto-oxidative action, such as described, for example, as malonyl derivatives in “Makromolekulare Chemie” 99 (1966), 96-102. Dibenzoyl peroxide and/or malonyl derivatives are preferably used.

As curing agents (catalysts) for the photopolymerisation there can be used, for example, benzophenone and derivatives thereof and also benzoin and its derivatives or mixtures thereof. More especially preferred photosensitisers are diketones such as 9,10-phenanthrenequinone, diacetyl, furil, anisil, 4,4′-dichlorobenzil and 4,4′-dialkoxybenzil, camphorquinone or mixtures thereof. Special preference is given to the use of camphorquinone. The use of one or more photosensitisers together with a reducing agent is preferred. Examples of reducing agents are amines such as cyanoethyl-methylaniline, N,N-dimethyl-sym.-xylidine and N,N-3,5-tetramethylaniline and 4-dimethylaminobenzoic acid esters or mixtures thereof.

The root filling materials preferably contain dual-curing curing systems, that is to say they are cold-curing and contain in addition catalysts for photo-curing.

The amount of polymerisation catalysts in the dental material is generally from 0.001 to 5% by weight, preferably from 0.1 to 3% by weight.

The root filling material is preferably produced in the form of a two-component material which, after mixing, fully cures in the cold state and is additionally photopolymerisable. The catalysts of the above-described initiator systems are incorporated into separate pastes. By mixing the two pastes together there is obtained a composition which provides a sufficiently long processing time and then slowly cures fully. The surface can be photopolymerised at an early stage so that it is possible immediately to continue working above the filling with other materials, for example with methacrylate composites for core build-ups or with customary filling composites. In the presence of a suitable irradiation device that penetrates sufficiently far into the root canal, formulations in the form of a one-component paste are also suitable, however.

As further component, the compositions according to the invention contain X-ray-opacifying additives, for example in the form of microfine metal powders from the group zinc, ytterbium, yttrium, gadolinium, zirconium, titanium, strontium, tungsten, tantalum, niobium, barium, bismuth, molybdenum and tantalum or alloys thereof, and/or in the form of their oxides, fluorides, sulfates, phosphates, silicates, carbonates, tungstates and carbides and/or in the form of mixtures thereof or as described, for example, in U.S. Pat. No. 3,925,895.

The X-ray-opacifying additive(s) according to the invention are present in the range of from 10 to 70% by weight, preferably from 30 to 50% by weight.

Preferably the surfaces of the X-ray-opacifying additives and of the further auxiliaries (such as, for example, inert fillers, ion-donating fillers, thixotropic filler additives, disinfectant additives, acids and structure formers) should not be treated with a coupling agent (cross-linking agent) which increases the bond between the filler and the polymerisable resin. Especially, the surfaces of the X-ray-opacifying additives are not treated with a silanising agent. It is especially preferred that the surfaces of the X-ray-opacifying additives according to the invention are not treated with, for example, trimethylsilane, methacryloxypropyltrimethoxysilane.

Furthermore, the root filling material according to the invention can also contain customary auxiliaries such as, especially, inert fillers such as, for example, dental glasses, quartz or aluminium oxide and/or ion-donating inorganic fillers and/or thixotropic filler additives, preferably silicic acid. Such fillers are not silanised, however, for the purpose of the desired weaker mechanical properties of the root canal filling material. It is also possible, however, in addition for non-(meth)acrylate-group-containing liquid, solid or wax-like additives such as, for example, silicone oil, paraffin oil, Vaseline, wax, allyl-group-containing compounds, saturated or unsaturated esters of dibasic or polybasic acids to be present, provided that they are all sufficiently insoluble in the reacted or unreacted state.

Furthermore, the filler can also consist of prepolymerised filled and/or unfilled resins according to the invention. Preferably, the root canal filling material is prepolymerised and ground and then used as filler in the root canal filling material.

It is also possible for disinfectant additives such as, for example, benzalkonium chloride, chlorohexidine, nanosilver or nanocopper or devitalising additives to be present.

As ion-donating additive fillers there may be present, for example, calcium, strontium or zinc glasses or oxides and/or carbonate-, fluoride- or phosphate-containing additives. Examples of ion-donating glasses are, for example, glass powders as used in glass ionomer cements. Calcium-containing additives are, for example, calcium carbonate, tricalcium carbonate and tetracalcium phosphate, calcium hydroxyapatite, Ca-/Sr-apatite, fluoroapatite or apatites as described in DE 102004025030 A1. As zinc-containing additive there can preferably be used zinc oxide.

Furthermore, the compositions according to the invention can contain at least one bioactive or remineralising additive such as calcium hydroxide, calcium oxide, calcium fluoride, hydroxyapatite, fluoroapatite or one or more other apatites.

Especially preferably, the surfaces of the ion-donating additive fillers are not silanised.

It is also possible for polymerisable or non-polymerisable acids additionally to be present, as are customary in dental adhesives, dental compomers or dental resin-modified cements, such as, for example, methacrylated or non-methacrylated esters of phosphoric, phosphonic or carboxylic acids and of polyphosphonic or polycarboxylic acids.

Those additives can improve adhesion to the root canal wall and/or the migration of ions.

As structure formers, amorphous silicon dioxide modifications are advantageous. Examples are pyrogenic and precipitated silicic acids and also kieselguhr. Agglomerated pyrogenic silicic acids or sintered silica gel, as described in EP 0 040 232 and EP 0 113 926 (for example methyl-silanised pyrogenic silicic acid), are especially suitable. Those fillers have only a slight tendency towards post-thickening and have the property that their thixotropic behaviour is uniform and has very little dependency upon how quickly the processing takes place.

The root canal filling materials according to the invention are novel and extremely advantageous. They can be introduced, for example, into application capsules, mixed in commercially available capsule mixing apparatus and applied directly into the root canal using a suitable attached application cannula. They can, however, also be filled into dual cartridges or dual syringes and mixed using attached mixing cannulas and introduced directly into the root canal. They can be adjusted for sufficiently low viscosity and sufficiently long processing time. Sufficient X-ray opacity allows accurate placement of any gutta-percha point that may have been introduced and of the root filling material.

After fully curing, the strength is sufficient to bear the necessary stresses of, where applicable, a core build-up post or stresses caused by chewing. The root fillings, by virtue of their low degree of shrinkage and balanced low water absorption, are swollen hardly at all or only by a few percent by volume and, by virtue of their high quality of sealing, are totally bacteria-tight. The polymerisation shrinkage that occurs can thus be compensated. The water absorption is determined in accordance with the standard ISO 4049.

The compositions according to the invention exhibit a significantly lower compressive strength, which is determined in accordance with the standard ISO 9917-1, of less than 50 MPa, especially less than 25 MPa, and accordingly significantly less than normal filling composite preparations (with usually significantly more than 200 MPa); furthermore, unlike the latter, they exhibit a high degree of deformability and ability to be cut. As a result, the compositions according to the invention, used as root filling materials, in the case of apical problems at the apex of the root can be removed at any time using a root canal fraise easily and without piercing the walls of the root canal. That is advantageous because the roots of teeth are often substantially curved. Once the problem has been eliminated, the root canal can be closed again without problems using fresh material according to the invention.

The present invention relates especially to a polymerisable composition containing
a) from 10 to 50% by weight, especially from 15 to 30% by weight, of at least one higher molecular weight di(meth)acrylate,
b) at least one curing agent, and
b) from 10 to 70% by weight, preferably from 30 to 50% by weight, of at least one X-ray-opacifying additive.

Especially, the surfaces of X-ray-opacifying additives are not treated with a coupling agent (cross-linking agent) which increases the binding between the filler and the polymerisable resin. Especially preferably, the surfaces of the X-ray-opacifying additives are not silanised.

The compositions according to the invention exhibit very low solubility and are very well tolerated. For example, the compositions according to the invention can remain in the root canal as root filling material temporarily (for example for a few days), semi-permanently (for a period of months) or permanently.

Adjusted with shorter setting times, the compositions according to the invention are also excellently suitable for use as a provisional cement for fixing crowns and bridges and for use as a material for provisional fillings of cavities. The materials provide a bacteria-tight seal and can be removed again easily and without damaging the prepared tooth structures.

Confirmation that the provisional fillings or cements have been properly removed can easily be obtained by X-ray diagnosis.

### COMPARISON EXAMPLE 1

Two pastes are stirred:

| | parts by weight |
|---|---|
| Paste A | |
| hexanediol dimethacrylate | 10 |
| tetraethylene glycol | 19 |
| dimethacrylate (short chain) | |
| ethoxylated bisphenol A | 19 |
| dimethacrylate (short chain) | |
| dimethyl-p-toluidine | 0.3 |
| methyl-silanised pyrogenic silicic acid | 3 |
| barium glass, methacrylsilanised | 48 |
| Paste B | |
| hexanediol dimethacrylate | 10 |
| tetraethylene glycol | 19 |
| dimethacrylate (short chain) | |
| ethoxylated bisphenol A | 19 |
| dimethacrylate (short chain) | |
| dibenzoyl peroxide | 0.5 |
| methyl-silanised pyrogenic silicic acid | 3 |
| barium glass, methacrylsilanised | 48 |

Both pastes have good flowability. After mixing, they cure with a processing time of 8 min and a hardening time of 20 min. After 24 h the following properties are measured:

| | |
|---|---|
| compressive strength | 218 MPa |
| flexural strength | 66 MPa |
| elasticity modulus | 2540 MPa |

The compressive strength is calculated in accordance with ISO standard 9917-1; the flexural strength and the elasticity modulus are determined in accordance with ISO 4049.

The fully cured material is very hard and cannot be removed from a root canal using a file or a hand instrument. If mechanical drilling-out is necessary, it is not possible to distinguish sufficiently well between the filling and the wall of the root canal. The X-ray visibility, corresponding to 1.9 mm of aluminium, is not sufficient for clear diagnosis in the root canal. After several weeks' storage in water, there are visible marginal gaps between the filling and the wall of the root canal. Bacterial tightness cannot be ensured.

### COMPARISON EXAMPLE 2

Two pastes are stirred:

| | parts |
|---|---|
| Paste A | |
| hexanediol dimethacrylate | 11 |
| tetraethylene glycol | 21 |
| dimethacrylate (short chain) | |
| ethoxylated bisphenol A | 21 |
| dimethacrylate (2-4)BADMA (short chain) | |
| dimethyl-p-toluidine | 0.3 |
| methyl-silanised pyrogenic silicic acid | 3 |
| zirconium dioxide, unsilanised | 28 |
| zinc oxide, unsilanised | 16 |
| Paste B | |
| hexanediol dimethacrylate | 11 |
| tetraethylene glycol | 21 |
| dimethacrylate (short chain) | |
| ethoxylated bisphenol A | 21 |
| dimethacrylate (2-4)BADMA (short chain) | |
| dibenzoyl peroxide | 0.5 |
| methyl-silanised pyrogenic silicic acid | 3 |
| zirconium dioxide, unsilanised | 28 |
| zinc oxide, unsilanised | 16 |

| | |
|---|---|
| compressive strength | 296 MPa |
| flexural strength | 69 MPa |
| elasticity modulus | 1750 MPa |

### EXAMPLE

### Example According to the Invention

Two pastes are stirred:

| | parts by weight |
|---|---|
| Paste A | |
| hexanediol dimethacrylate | 11 |
| polyethylene glycol 600 | 21 |
| dimethacrylate (long chain) | |
| ethoxylated bisphenol A | 21 |
| dimethacrylate E(30)BADMA (long chain) | |
| dimethyl-p-toluidine | 0.3 |
| methyl-silanised pyrogenic silicic acid | 3 |
| zirconium dioxide, unsilanised | 28 |
| zinc oxide, unsilanised | 16 |
| Paste B | |
| hexanediol dimethacrylate | 11 |
| polyethylene glycol 600 | 21 |
| dimethacrylate (long chain) | |
| ethoxylated bisphenol A | 21 |
| dimethacrylate E(30)BADMA (long chain) | |
| dibenzoyl peroxide | 0.5 |
| methyl-silanised pyrogenic silicic acid | 3 |
| zirconium dioxide, unsilanised | 28 |
| zinc oxide, unsilanised | 16 |

Both pastes have good flowability. After mixing, they cure with a processing time of 12 min and a hardening time of 30 min. After 24 h the following properties are measured:

| | |
|---|---|
| compressive strength | 38 MPa |
| flexural strength | 8 MPa |
| elasticity modulus | 92 MPa |

The fully cured material is strong, but can readily be removed from a cavity using a file or a hand instrument. If mechanical drilling-out is necessary, it is easily possible to distinguish between the filling and the wall of the root canal. The X-ray visibility, corresponding to 5.5 mm of aluminium, is excellent for diagnosis in the root canal. After several weeks' storage in water, no marginal gaps can be found between the filling and the root canal wall. Bacterial tightness can be ensured.

The composition of the present invention and the resulting polymerisation product have the following advantageous properties:
the composition is thinly liquid
as a result of the non-surface-treated fillers, relatively weak mechanical properties are achieved (for example compressive strength of <50 MPa)
a very high degree of X-ray visibility of >400% Al is achieved
easy frangibility
the product is removable manually using a root canal file
ability to be cut
volumetric stability, less shrinkage caused by water absorption

1. Polymerisable composition containing
at least one higher molecular weight di(meth)acrylate,
at least one curing agent, and
at least one X-ray-opacifying additive.

2. Polymerisable composition according to claim 1, characterised in that the higher molecular weight di(meth)acrylate has an average molecular weight of more than 600.

3. Polymerisable composition according to claim 1 or 2, characterised in that the higher molecular weight di(meth)acrylate contains at least two hydrophilic units.

4. Polymerisable composition according to any one of claims 1 to 3, characterised in that the higher molecular weight di(meth)acrylate contains at least 10 hydrophilic units.

5. Polymerisable composition according to any one of claims 1 to 3, characterised in that the higher molecular weight di(meth)acrylate contains at least 20 hydrophilic units.

6. Polymerisable composition according to any one of claims 1 to 3, characterised in that the higher molecular weight di(meth)acrylate contains at least 30 hydrophilic units.

7. Polymerisable composition according to any one of claims 3 to 6, characterised in that the hydrophilic units are —CH₂—CH₂—O— units.

8. Polymerisable composition according to any one of claims 1 to 7, characterised in that the higher molecular weight di(meth)acrylate having hydrophilic units is a polyethylene glycol di(meth)acrylate.

9. Polymerisable composition according to any one of claims 1 to 7, characterised in that the higher molecular weight di(meth)acrylate having hydrophilic units is a polyethoxylated bisphenol A di(meth)acrylate.

10. Polymerisable composition according to any one of claims 1 to 9, characterised in that it contains the higher molecular weight di(meth)acrylate in an amount of from 2 to 90% by weight.

11. Polymerisable composition according to any one of claims 1 to 9, characterised in that it contains the higher molecular weight di(meth)acrylate in an amount of from 10 to 50% by weight.

12. Polymerisable composition according to any one of claims 1 to 9, characterised in that it contains the higher molecular weight di(meth)acrylate in an amount of from 15 to 30% by weight.

13. Polymerisable composition according to any one of claims 1 to 12, characterised in that it additionally contains at least one low molecular weight di(meth)acrylate having an average molecular weight of less than 600.

14. Polymerisable composition according to claim 13, characterised in that it contains the low molecular weight di(meth)acrylate in an amount of less than 30% by weight.

15. Polymerisable composition according to claim 13, characterised in that it contains the low molecular weight di(meth)acrylate in an amount of less than 18% by weight.

16. Polymerisable composition according to any one of claims 1 to 15, characterised in that the curing agent is a free-radical-generating polymerisation system.

17. Polymerisable composition according to claim 16, characterised in that the free-radical-generating polymerisation system contains a peroxide.

18. Polymerisable composition according to claim 16 or 17, characterised in that the free-radical-generating polymerisation system contains a malonyl derivative.

19. Polymerisable composition according to any one of claims 16 to 18, characterised in that the free-radical-generating polymerisation system contains a photoinitiator.

20. Polymerisable composition according to claim 19, characterised in that the free-radical-generating polymerisation system contains camphorquinone.

21. Polymerisable composition according to any one of claims 1 to 20, characterised in that it additionally contains non-(meth)acryl-containing liquid additives.

22. Polymerisable composition according to any one of claims 1 to 21, characterised in that the X-ray-opacifying additive is selected from the group comprising zinc, ytterbium, yttrium, gadolinium, zirconium, titanium, strontium, tungsten, tantalum, niobium, barium, bismuth, molybedenum and tantalum powders, pulverulent alloys thereof, oxides, fluorides, sulfates, carbonates, tungstates and carbides thereof, and mixtures thereof.

23. Polymerisable composition according to any one of claims 1 to 22, characterised in that it contains consistency- or strength-modifying additives such as poly(lactides), poly(glycolides), poly(lactide-co-glycolides), poly(methacrylates), amorphous partially methylsilanised silicic acids, unsilanised powders of quartz, silicate glass, glass fibres, barium silicates, strontium silicates, borosilicates, lithium silicates, aluminium oxides, zinc oxide or titanium oxide or mixtures of the above-mentioned additives.

24. Polymerisable composition according to any one of claims 1 to 23, characterised in that it contains at least one bioactive or remineralising additive such as calcium hydroxide, calcium oxide, calcium fluoride, hydroxyapatite, fluoroapatite or one or more other apatites.

25. Polymerisable composition according to any one of claims 1 to 25, characterised in that it contains at least one bacteriostatic or bactericidal additive such as benzalkonium chloride, chlorohexidine, nanosilver or nanocopper.

26. Polymerisable composition according to any one of claims 1 to 25, characterised in that it contains at least one polymerisable or non-polymerisable acid.

27. Polymerisable composition according to claim 26, characterised in that the polymerisable or non-polymerisable acid is a phosphoric, phosphonic or carboxylic acid.

28. Use of a polymerisable composition according to any one of claims 1 to 27 for filling and sealing root canals.

29. Use of a polymerisable composition according to claim 28, characterised in that the polymerisable composition, after polymerisation, forms a strong sealing body having a compressive strength of less than 50 Mpa.

30. Use of a polymerisable composition according to any one of claims 1 to 27 in the provisional filling of tooth cavities.

31. Use of a polymerisable composition according to claim 30, characterised in that the polymerisable composition, after polymerisation, forms a strong provisional cavity filling having a compressive strength of less than 50 Mpa.

32. Use of a polymerisable composition according to any one of claims 1 to 27 as material for provisionally cementing crowns, bridges, inlays, onlays and veneers.

33. Use of a polymerisable composition according to claim 32, characterised in that the polymerisable composition, after polymerisation, forms a strong fixing cement having a compressive strength of less than 50 Mpa and the provisionally fixed crowns, bridges, inlays, onlays or veneers, on being removed, can be removed intact.
